(19) United States
(12) Patent Application Publication
(10) Pub. No.: US 2015/0351737 A1
(43) Pub. Date: Dec. 10, 2015

(54) MULTI-CHAMBERED CANNULA

(71) Applicant: Jeffrey Jackson, Holliday, UT (US)

(72) Inventor: Jeffrey Jackson, Holliday, UT (US)

(21) Appl. No.: 14/492,977

(22) Filed: Sep. 22, 2014

Related U.S. Application Data
(60) Provisional application No. 62/008,397, filed on Jun. 5, 2014.

(51) Int. Cl. A61B 17/02

(52) U.S. Cl. CPC A61B 17/0293

(57) ABSTRACT

A cannula including multiple separated compartments is disclosed. The multiple compartments enable a surgeon to pass multiple surgical tools through the cannula without those tools contacting or becoming entangled with each other. The cannula and its compartments may be customized for particular surgical operations and/or particular surgical tools.

### RELATED APPLICATIONS

This application claims the benefit of U.S. Provisional Patent Application No. 62/008,397, entitled Double Barrel Cannula and filed on Jun. 5, 2014, the contents of which are incorporated by reference herein for all purposes.

### TECHNICAL FIELD

Embodiments of the present invention relate to medical devices and methods for accessing an anatomical space of the body. More specifically, embodiments of the invention relate to compartmentalized cannulas that enable the separation of surgical tools within a single cannula.

### BACKGROUND

During arthroscopic and endoscopic surgery, cannulas are used to access joints and body cavities. Cannulas increase ease of instrument entry and removal without damage to the tissues. However, these cannulas typically include only a single channel through which surgical tools, such as surgical instruments, sutures, fiber optic cameras, etc., travel during surgical procedures. In such circumstances, the surgical tools may become entangled.

### SUMMARY

In an exemplary embodiment of the present disclosure, a cannula includes two or more separated compartments or channels. Each compartment is dimensioned to accommodate particular surgical tools, such as instruments, sutures, fiber optic cameras, etc. The compartments are isolated from each other so that the surgical components remain separated as they pass through the compartments. In some embodiments, one or more of the compartments are partially closed compartments in which the surgical tools may be stored for easy access during an operation.

For example, in one embodiment, a multi-chambered cannula for simultaneously receiving a first surgical tool of a plurality of surgical tools and a second surgical tool of the plurality of surgical tools comprises a top portion; a bottom portion; a first chamber extending between the top portion and the bottom portion, the first chamber being a hollow passage to receive the first surgical tool through the first chamber; a second chamber extending between the top portion and the bottom portion, the second chamber being a hollow passage to receive the second surgical tool through the second chamber; and a dividing wall extending from the bottom portion towards the top portion, the dividing wall at least partially separating the first chamber from the second chamber to enable the first surgical tool to pass through the first chamber while the second surgical tool passes simultaneously and separately through the second chamber.

The multi-chambered cannula above further comprises a dam, which may be incorporated into the top portion of the cannula and extend over the first chamber. The dam may further extend over the second chamber.

In the multi-chambered cannula above, the dividing wall extends upwards from the bottom portion of the cannula to create a seal with a lower surface of the dam.

In the multi-chambered cannula above, the first chamber has a first cross-section and the second chamber has a second cross-section, the first cross-section and the second cross-section being non-congruent.

In the multi-chambered cannula above, a shape of a concave surface of the second chamber matches a shape of a convex surface of the first chamber.

In the multi-chambered cannula above, the first chamber has a larger cross-sectional size than a cross-sectional size of the second chamber.

In the multi-chambered cannula above, a dam that includes a flexible opening configured to resile against a surgical instrument inserted through the flexible opening to create a seal.

In the multi-chambered cannula above, a portal is in fluid communication with the first chamber and with the second chamber.

In the multi-chambered cannula above, the portal is in fluid communication with the first chamber and with the second chamber through an aperture, and the dividing wall is located adjacent to the aperture to divide fluid passing through the aperture into the first chamber and into the second chamber.

In another exemplary embodiment, a multi-chambered cannula for receiving a first surgical tool of a plurality of surgical tools and a second surgical tool of the plurality of surgical tools comprises an outer wall unitarily formed of a single material that defines a passage between a top surface of a cannula and a bottom surface of the cannula; a first channel within the passage, the first channel being configured to enable the first surgical tool to pass through the first channel; a second channel within the passage, the second channel being configured to enable the second surgical tool to pass through the second channel; and a dividing wall within the passage that at least partially isolates the first channel from the second channel in order to enable the separate and simultaneous passing of the first surgical tool through the first channel and the second surgical tool through the second channel.

In the multi-chambered cannula above, the first channel is configured to receive a suture through the first channel, the first channel has a cross-sectional area, the second channel has a cross-sectional area that is larger than the cross-sectional area of the first channel, and a width of the cannula measured between opposing sections of an inner surface of the cannula is between 4 and 30 mm.

In the multi-chambered cannula above, the width of the cannula is between 4 and 6 mm.

In the multi-chambered cannula above, the cannula further comprises a third channel within the passage, the third chamber being configured to enable the surgeon to store a third surgical tool in the third chamber.

In another embodiment, a medical device configured to simultaneously accommodate sutures and other surgical instruments comprises a cannula formed of a resilient material, the cannula including a channel extending between a top surface of the cannula and a bottom surface of the cannula; and a dividing wall placed within the channel to divide the channel into a first compartment and a second compartment, wherein the first compartment has a first cross-sectional size configured to accommodate sutures and the second compartment has a second cross-sectional size configured to accommodate another surgical instrument.

In the medical device above, the medical device includes a dam located proximate to the top surface of the cannula.

In the medical device above, the dam seals an upper portion of both the first and second compartments.

In the medical device above, the dam is formed of a resilient material and includes a first perforation located above the first compartment and a second perforation located above the second compartment, and wherein the dam is configured to resile against surgical instruments placed through the first and second perforations to create seals.

In the medical device above, the first compartment has a first cross-sectional area and the second compartment has a second cross-sectional area, and a ratio of the first cross-sectional area to the second cross-sectional area ranges from 1:1 to 20:1.

In the medical device above, a cross-sectional profile of the second chamber is configured to match a particular surgical instrument.

In the medical device above, the dividing wall is a removeable dividing wall adapted to be selectively secured to the cannula.

In the medical device above, an outer surface of the cannula has an outer profile configured to continuously contact an outer surface of a surgical incision in order to reduce fluid loss along the outer surface of the cannula.

In the medical device above, the first compartment forms a blind passage.

In another embodiment, a multi-chambered cannula for simultaneously receiving a first tool of a plurality of surgical tools and a second tool of a plurality of surgical tools includes a top portion; a bottom portion; a first chamber extending between the top portion and the bottom portion, the first chamber being a hollow passage to receive the first surgical tool through the first chamber; a second chamber extending between the top portion and the bottom portion, the second chamber being a hollow passage to receive the second surgical tool through the second chamber; and a means for separating the first chamber from the second chamber to enable the first surgical tool to pass through the first chamber while the second surgical tool passes simultaneously and separately through the second chamber.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a perspective view of an exemplary cannula that includes two separate compartments.

FIGS. 2A-2F illustrate bottom views of exemplary cannulas with multiple compartments.

FIG. 3 illustrates a perspective view of an exemplary cannula that includes two separate compartments.

FIG. 3A illustrates a cut-away view of the cannula of FIG. 3, cut along the lines 3A-3A in FIG. 3.

FIG. 4 illustrates a perspective view of an exemplary cannula formed by merging two cylindrical components.

FIG. 4A illustrates a cut-away view of the cannula of FIG. 4, cut along the lines 4A-4A in FIG. 4.

FIG. 5 illustrates a perspective view of an exemplary oblong cannula that includes two separate compartments.

FIG. 5A illustrates a cut-away view of the cannula of FIG. 5, cut along the lines 5A-5A in FIG. 5.

FIG. 6 illustrates a cut-away view of a cannula in which two separate instruments are simultaneously passed through the chambers.

FIG. 7 illustrates a cut-away view of an exemplary cannula in which a portal directly communicates with two compartments within the cannula.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

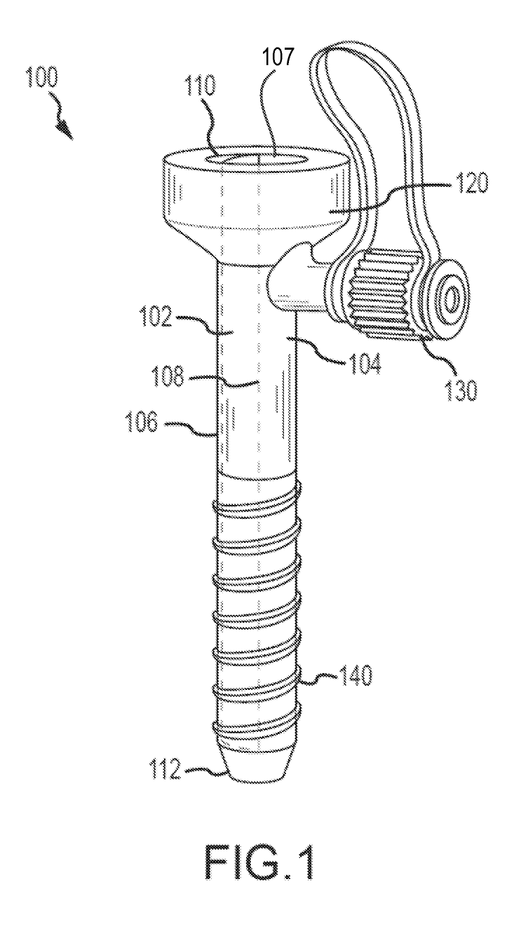

According to some embodiments of the present invention, a double barrel cannula includes multiple chambers through which surgical tools, such as surgical instruments, sutures, fiber optic cameras, etc., travel during surgical procedures. For example, as shown in FIG. 1, a cannula 100 includes a first chamber 102 (also referred to as a first channel or first barrel) and a second chamber 104 (also referred to as a second channel or second barrel). Each chamber 102, 104 is bound by the outer wall 106 of the cannula 100 (specifically an inner surface 107 of the outer wall 106) and by a dividing wall 108 within the cannula 100. In the embodiments shown in FIG. 1, the chambers 102, 104 are each open at a top end 110 of the cannula 100 and at a bottom end 112 of the cannula 100 to define passageways through the cannula 100.

The particular dimensions of the cannula 100, including the dimensions and number of chambers in the cannula, may vary depending on the surgical technique to be employed, the joint or body cavity that is being operated on, and/or the surgical tools that will be used during the operation. For example, one compartment may have a smaller diameter or a particular configuration (e.g., a particular cross-sectional profile) to fulfill a specific role (e.g., passing sutures), while the other compartment may be larger or of a different configuration to fulfill a different role (e.g., passing a larger instrument).

In some embodiments, one of the passages, e.g., the second passage 104, is closed at or near the bottom end 112 of the cannula 100. This creates an enclosure or blind passage in which surgical tools, such as sutures, may be stored for easy access during surgery.

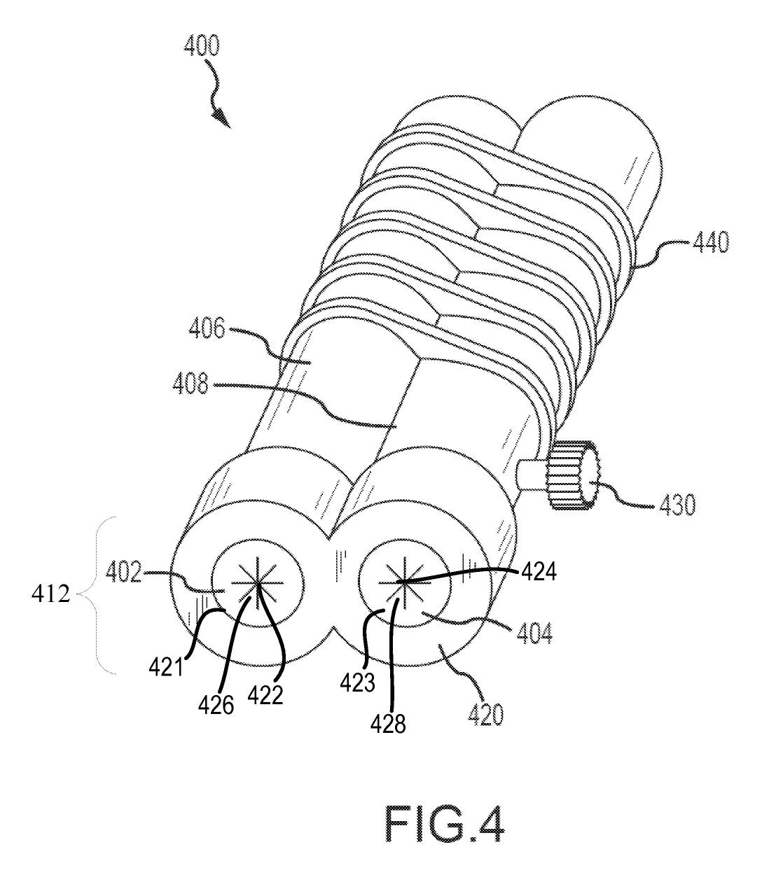

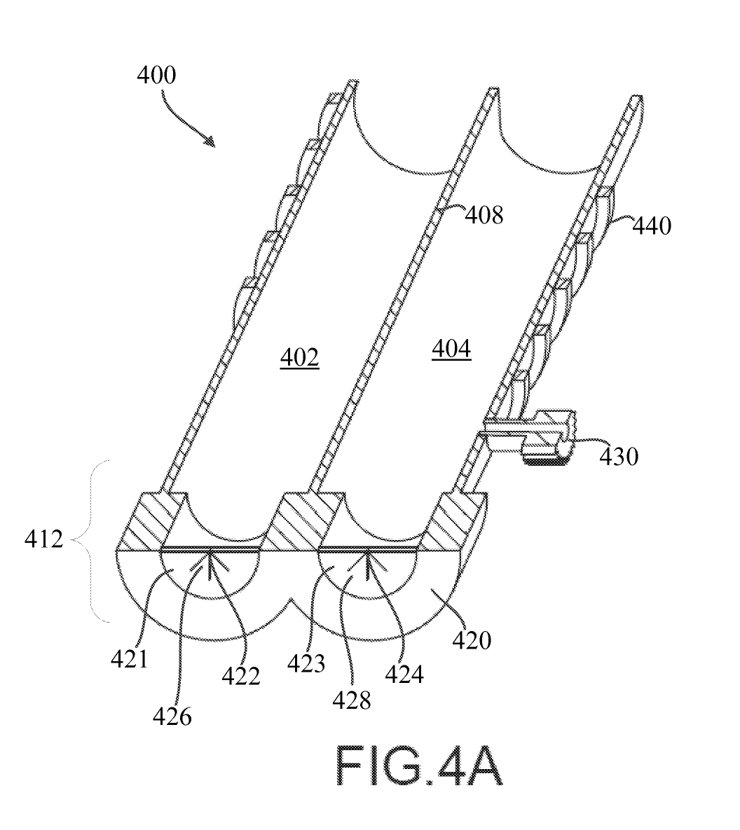

The cannula 100 may also incorporate a dam (e.g., dam 421 in FIG. 4) that prevents egress of fluid from the body and the cannula. The dam may be incorporated into an enlarged portion 120 of the cannula 100, and may be placed in the interior of the cannula (e.g., within the outer wall 106 of the cannula 100) to create a seal with the inner surface 107 of the outer wall 106. A single dam may span the compartments in the cannula (e.g., compartments 102, 104) to prevent egress of fluid from each compartment. In some embodiments, the dividing wall 108 terminates at a lower surface of the dam, and may contact that lower surface to create a seal. In other embodiments, the dividing wall terminates below the lower surface of the dam. In some embodiments, each compartment terminates with a separate dam, for example, two separate dams for two separate compartments (e.g., dams 421 and 423 in FIG. 4). The dam or dams can also be positioned anywhere along the longitudinal length of the cannula, though in many embodiments each dam will be located at or near the proximal or top end 110 of the cannula 110.

As also shown in FIG. 1, the cannula 100 includes a portal 130, also referred to as an inflow/outflow portal 130. This portal 130 enables the inflow of saline, water, air, etc. to the surgical site. For example, arthroscopic surgery physicians often use a saline solution that continually runs in and out of the joint throughout the surgery. In those cases, one cannula is designated as the inflow portal and another cannula is designated as the outflow portal. The portal 130 of the cannula used as the inflow portal couples to a saline pump via a tube so that saline is pumped through the tube and into the cannula. With respect to the cannula 100 shown in FIG. 1, the portal 130 may connect to one or both compartments 102, 104. Thus, the portal 130 may be in fluid communication with one or all of the channels in the cannula 100. In some embodiments, the cannula 100 includes threads or flanges 140 that prevent the inadvertent removal of the cannula 100 from the body. In other embodiments, the cannula 100 includes deployable wings and/or surface textures (e.g., rough or ribbed textures) to prevent inadvertent removal of the cannula 100. In some embodiments, the wings are inflatable wings or are resilient wings.

The cannula 100 may be constructed of various types of materials including, but limited to, rubber, plastics, thermoplastics, Teflon, or various metals including copper, aluminum, or stainless steel. The cannula 100, including some or all of its components, may be unitarily or integrally formed of one or more materials. As mentioned above, the outer surface of the cannula can be rough, smooth, threaded, ribbed, or have one or more deployable wings.

The dividing wall 108 may be formed of various materials. For example, in some embodiments the dividing wall 108 is formed of a resilient material, while in other embodiments the dividing wall 108 is formed of a rigid material. In some embodiments, the dividing wall 108 is fluid impermeable, while in other embodiments the dividing wall 108 is formed of a mesh or a perforated material so that fluids may pass from one chamber to another while the surgical tools within the chambers remain isolated. While in some embodiments the dividing wall 108 extends the full longitudinal length of the cannula, in other embodiments the dividing wall 108 does not extend the full longitudinal length of the cannula. In yet other embodiments, the dividing wall 108 includes distinct sections each spanning a portion of the longitudinal length of the cannula. These distinct sections may be vertically or longitudinally aligned within the cannula 100.

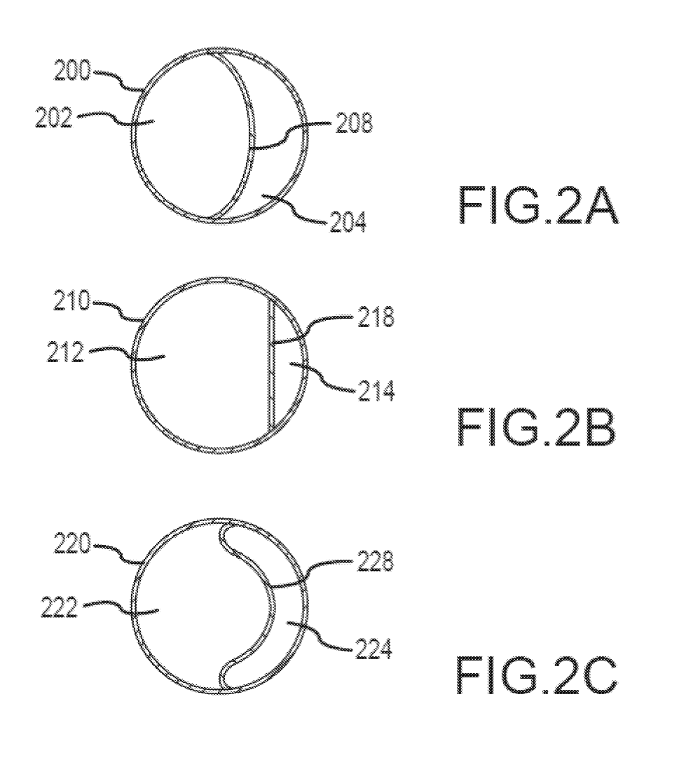

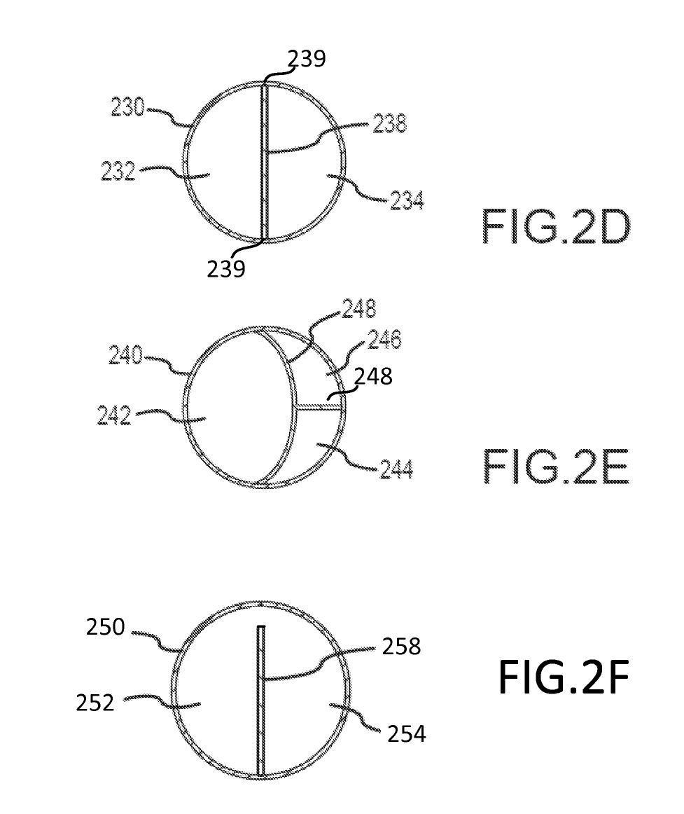

As shown in FIGS. 2A-2F, the relative sizes, shapes, and number of compartments within a cannula may vary. For example, in FIG. 2A, a cannula 200 includes a first chamber 202 and a second chamber 204 separated by a dividing wall 208. In those embodiments, the first chamber 202 is convex and is larger than the concave second chamber 204, and the dividing wall 208 arcs across the cannula to form the boundary between the first chamber 202 and the second chamber 204. In FIG. 2B, a cannula 210 includes a first chamber 212 and a second chamber 214 separated by a dividing wall 218. As shown in that figure, the dividing wall 218 forms a straight boundary.

In FIG. 2C, a cannula 220 includes a first chamber 222 and a second chamber 224 separated by a dividing wall 228. In these embodiments, the dividing wall 228 forms a customized boundary that may be configured to accommodate or match the dimensions of particular surgical components. In FIG. 2D, a cannula 230 includes a first chamber 232 and a second chamber 234 separated by a dividing wall 238. The dividing wall is a removeable dividing wall and is secured within the cannula through grooves 239. In FIG. 2E, a cannula 240 includes a first chamber 242, a second chamber 244, and a third chamber 246 that are each separated by dividing walls 248. In FIG. 2F, a cannula 250 includes a first chamber 252 and a second chamber 254 that are partially separated in a lateral direction by a dividing wall 258.

The thickness of the dividing wall (e.g., dividing wall 238) may vary according to particular environments. For example, the wall 238 may be thicker to prevent incidental puncture by, e.g., a scalpel as it passes through the first chamber 232. In other embodiments, the wall 238 may be thinner to increase the cross sectional size of the chambers 232, 234 without increasing the cross sectional size of the cannula 230.

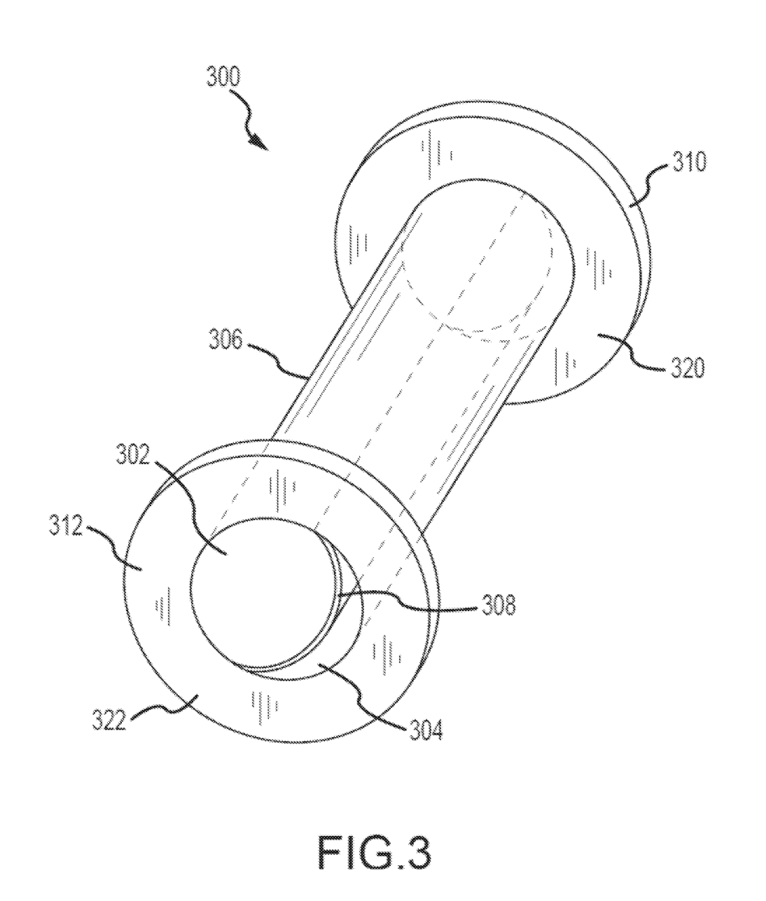

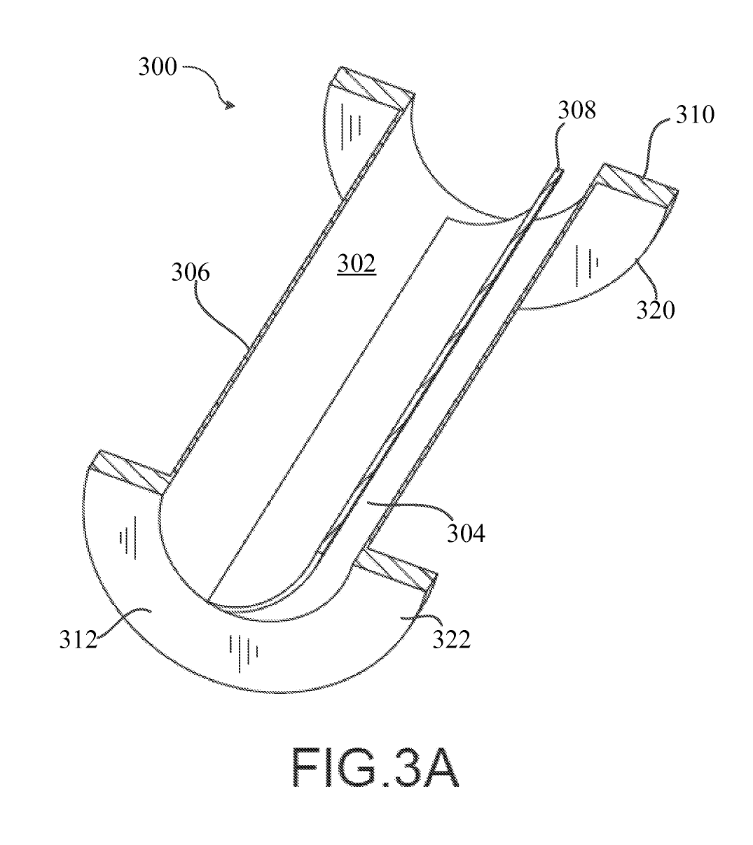

As shown in FIGS. 3 and 3A, a flexible cannula 300 includes a first chamber 302 and a second chamber 304. Each chamber 302, 304 is bound by the outer wall 306 of the cannula 300 and by a dividing wall 308. The chambers 302, 304 are each open at a top end 310 and at a bottom end 312 to define open passageways through the cannula 300. The cannula 300 also includes flanges 320, 322 that are placed at the top end 310 and the bottom end 312 of the cannula 300, respectively. These flanges 320, 322 hold the cannula 300 in position. The outer flange 322 sits on the skin and the inner flange 320 sits inside the capsule or joint space of the patient during surgery. In some embodiments, the flexible cannula 300 (including the flanges 320, 322 and the dividing wall 308) may be unitarily formed of a flexible material.

In some embodiments, and as shown in FIGS. 4 and 4A, a cannula 400 includes a first chamber 402 and a second chamber 404. The first chamber 402 and the second chamber 404 each have a circular cross-sectional diameter bound by the outer wall 406 and by a dividing wall 408. As shown in FIG. 4, the outer wall 406 and the dividing wall 408 are formed by merging two cylindrically shaped cannula portions. The top portion 412 of the cannula 400 incorporates an expanded section 420 in which two dams 421 and 423 are secured. Each dam 421, 423 has a flexible opening 422, 424 through which instruments may be inserted into the chambers 402, 404. The flexible openings 422, 424 are formed by a series of resilient flaps (e.g., flaps 426, 428). The resilient flaps 426, 428 resile against inserted instruments to seal the chambers 402, 404. While the resilient flaps 426, 428 in FIG. 4 have a triangular shape, other configurations may be used. For example, the resilient flaps may be configured to accommodate the dimensions of particular surgical tools. For example, if a camera has a circular cross-sectional shape, those portions of the resilient flaps that contact the camera may have a corresponding shape (e.g., concave and with a radius corresponding to the radius of the cross-sectional shape of the camera in order to form a better contact and seal with the camera). As also shown in FIGS. 4 and 4A, the cannula 400 includes a portal 430 and threads or flanges 440.

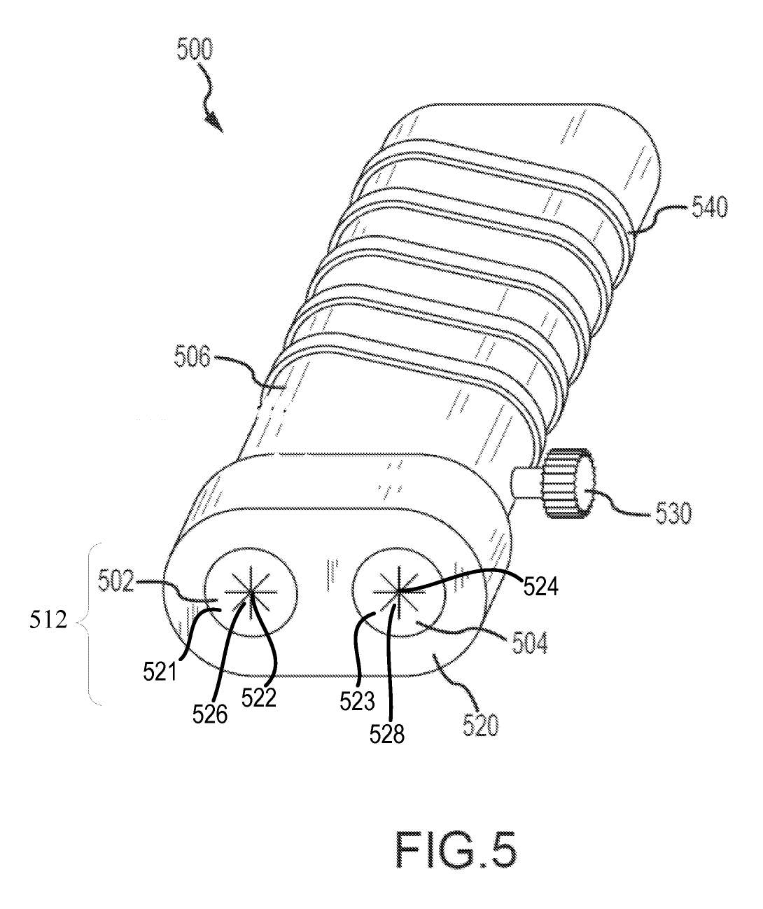

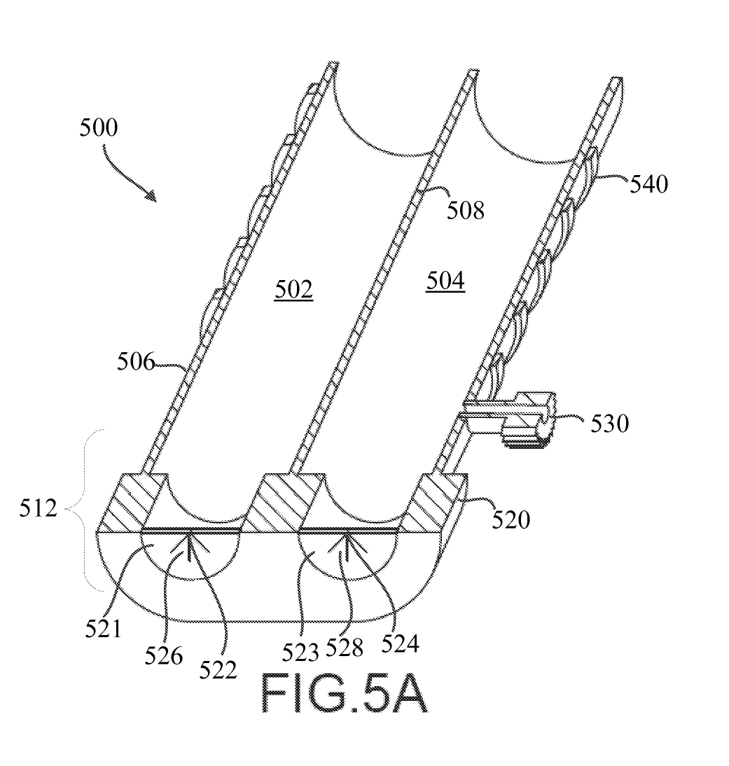

In some embodiments, and as shown in FIGS. 5 and 5A, a cannula 500 includes a first chamber 502 and a second chamber 504. The first chamber 502 and the second chamber 504 each have a circular cross-sectional diameter bound by the outer wall 506 and by a dividing wall 508 (shown in FIG. 5A). The outer wall 506 forms an oblong shape that engages the skin within the incision site to prevent fluids from escaping along the outer wall 506 of the cannula 500. The top portion 512 of the cannula 500 incorporates an expanded section 520 in which two dams 521 and 523 are secured. Each dam 521, 523 has a flexible opening 522, 524 through which instruments may be inserted into the chambers 502, 504. The flexible openings 522, 524 are formed by a series of resilient flaps 526, 528 similar to flaps 426, 428 discussed above. As also shown in FIGS. 5 and 5A, the cannula 500 includes a portal 530 and threads or flanges 540.

In some embodiments, the diameter of the cannula (e.g., cannula 100) is from 6 mm to 8 mm, though larger ranges are possible. For example, the diameter may range from 4 mm to 10 mm or larger, e.g., from 6 to 30 mm in diameter or greater. The diameter may be measured to include the width of the outer wall (e.g., outer wall 106) but in most embodiments refers to the distance between opposing portions of the inner surface of the outer wall (e.g., inner surface 107 of the outer wall 106). Thus, the diameter of the cannula, measured to include both chambers, may be smaller than two single-chamber cannulas together. The length of the cannula 100 is approximately 10 cm, but can range from 6 to 14 cm or greater.

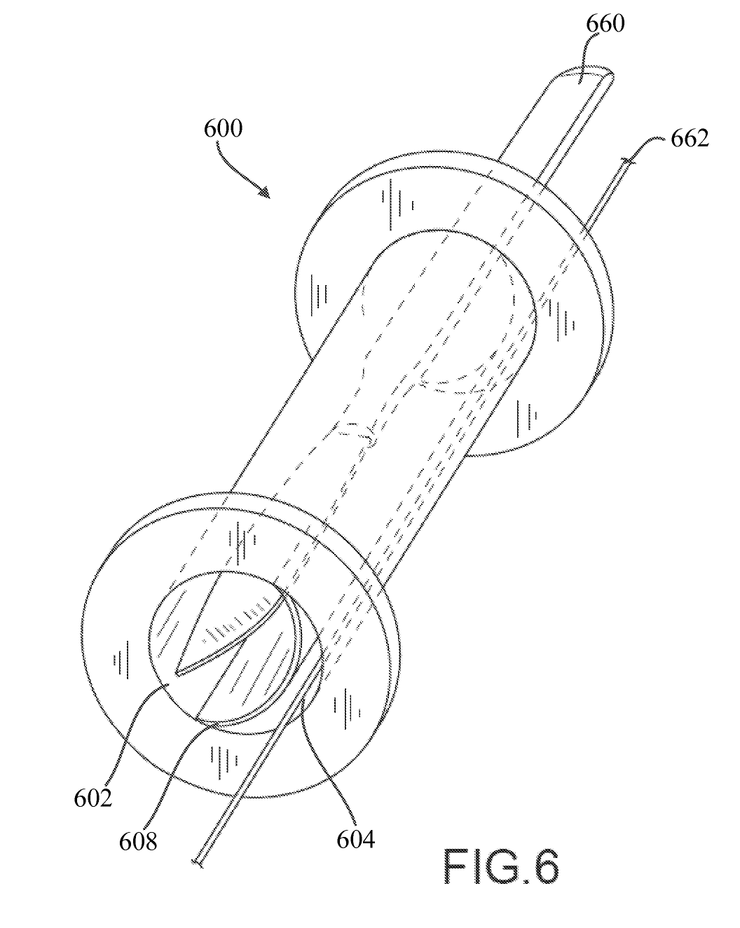

As shown in FIG. 6, a cannula 600 includes a first chamber 602 and a second chamber 604 separated by a dividing wall 608. The first chamber 602 is sized to receive a scalpel 660 through the first chamber 602. The second chamber 604 is sized to receive a suture 662 through the second chamber 604. The dividing wall 608 enables the simultaneous passage of the scalpel 660 and the suture 662 without the two surgical instruments contacting each other or becoming entangled.

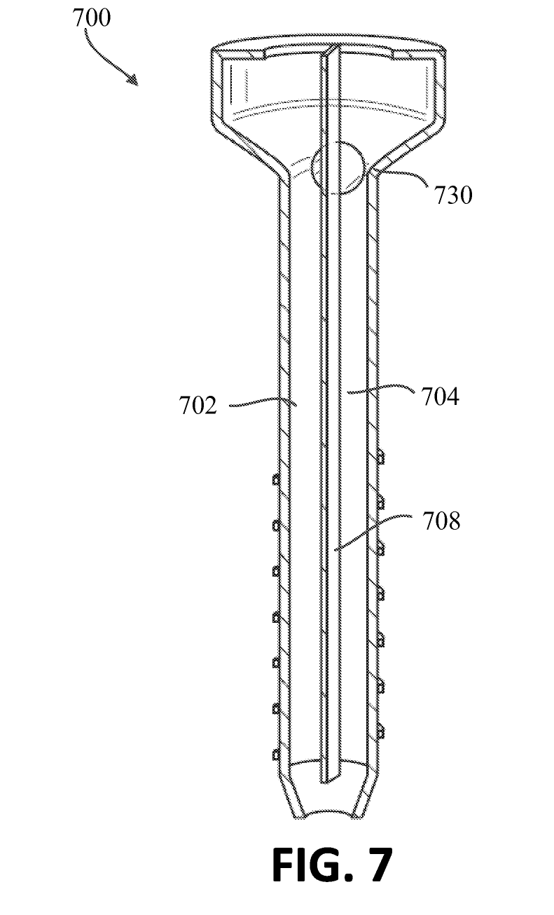

As shown in FIG. 7, a cannula 700 includes a first chamber 702 and a second chamber 704 separated by a dividing wall 708. The cannula 700 further includes a portal 730, similar to portal 130 in FIG. 1. The portal 730 is aligned with the dividing wall 708 to provide direct fluid access to both the first chamber 702 and the second chamber 704.

In some embodiments, the first chamber (e.g., chamber 202) may have a different cross-sectional size than the second chamber (e.g., chamber 204). For example, a cross-sectional width of the first chamber (measured at its widest point) may be 8 mm and the cross-sectional width of the second chamber (measured at its widest point) may be 6 mm. The ratio of cross-sectional widths may range from 1:1 to 5:1. In some embodiments, the first chamber may have a different perimeter length than the second chamber. For example, the perimeter of the first chamber may be 30 mm and the perimeter of the second chamber may be 15 mm. The ratio of perimeters may range from 1:1 to 15:1. In some embodiments, the first chamber may have a different cross-sectional area than the second chamber. For example, the cross sectional area of the first chamber may be 1.5 cm² and the cross sectional area of the second chamber may be 0.2 cm². The ratio of the cross sectional areas may range from 1:1 to 20:1.

As shown above, the cannula can have a round, oval or oblong, or figure-eight shaped cross section. The cross-sectional shape of the cannula may vary to accommodate various considerations. For example, a more compact cannula enables the surgeon to use a smaller incision. In addition, integrating two chambers into a single, compact cannula prevents fluid from escaping between the cannulas and creates greater support for the two chambers.

Several of the embodiments described above enable the passage of multiple separate surgical devices or implants without concern for entanglement or interference, which provides several specific advantages during surgical procedures. For example, an often-used technique in arthroscopic surgery is to place either a suture anchor with a suture or the suture alone in tissue by passing the suture anchor and/or suture through a cannula chamber into the body. The suture remains within the cannula chamber for later use or for tying. But, in order to remove the suture (e.g., so that the suture does not become entangled with other surgical tools), the cannula is removed from the body and then reinserted so that the suture is located outside the cannula. In some situations, this removal and exchange is performed several times during the surgery, resulting in tissue irritation and damage, as well as additional surgical time. Using a multi-chambered cannula, such as the cannula 100 in FIG. 1, however, enables the surgeon to successfully isolate the suture without removing the cannula.

Another advantage is that a multi-chambered cannula, e.g., the cannula 100 in FIG. 1, enables a surgeon to use two separate instruments in the same cannula. For example, the cannula 100 enables the surgeon to place a camera in one compartment of the cannula and to place another surgical instrument (e.g., a grasper) through the other compartment of the same cannula. With the use of single-compartment cannulas, in contrast, the surgeon is required to make another skin incision and portal, which may be located close to the first skin incision and portal if the same angle is required. Moreover, using the multi-chambered cannula, e.g., the cannula 100 in FIG. 1, enables the surgeon to use a smaller incision than would be required for two separate cannulas, as the cross-sectional size of the cannula 100 is significantly less than the cross sectional sizes of the two single cannulas placed together. Furthermore, the multi-chambered cannula, e.g., the cannula 100 in FIG. 1, creates more stability for the surgical passageways than two independently moveable single cannulas placed together.

In some embodiments, a multi-chambered cannula is formed using a removable dividing wall (e.g., wall 108 in FIG. 1) that is inserted into the cannula to create two separate chambers. The dividing wall may be secured within the cannula using grooves (e.g., grooves 239 in FIG. 2D) carved into the interior surface of the cannula, by frictional fitting, and/or by coupling the dividing wall to a dam. In other embodiments, an open tube is removeably secured within the cannula to create an additional and independent passage through the cannula. In some embodiments, a second, third, or fourth passage could be selectively added to and/or removed from a single compartment cannula. This allows for the additional compartments to be used in some cases and not others. In some embodiments, the second compartment could be added to or attached via either the top or the bottom of the cannula. In these embodiments, the additional compartment could either share a dam with the original compartment or could have a separate dam. The shape of the new compartment or compartments could vary as described above, e.g., with respect to FIGS. 2A-2F.

According to some embodiments, a multi-chambered cannula may be used in a variety of surgical procedures. For example, in some embodiments the multi-chambered cannula includes two chambers. The first chamber is closed near the bottom of the cannula to create a closed chamber in which surgical tools, such as sutures, are stored. The second chamber is open at the top and the bottom of the cannula to form a hollow passage. During surgery, a surgeon places the cannula into an incision in the patient to access the interior of the patient (e.g., a joint) through the second chamber. For example, the surgeon may pass a scalpel or other surgical tool through the second chamber to access the joint. When a suture is needed, the surgeon reaches into the first chamber and draws out one of the stored sutures. The surgeon then re-inserts the suture back into the cannula but now through the second chamber in order to access the joint. This enables the surgeon to reduce surgical time, as the sutures are already stored within the surgical site. In these embodiments, the first chamber may be have a smaller cross-section than the second chamber, since the first chamber simply stores sutures, which enables the second chamber to have a greater cross-section to allow larger instruments and more space for the surgeon.

In other embodiments, the multi-chambered cannula includes two chambers that are both open passages. During surgery, the surgeon uses both chambers to separately but simultaneously pass or use two different surgical tools. For example, the surgeon may place a camera in one chamber while passing and using another tool (e.g., a scalpel) through the other chamber. The dividing wall prevents the cutting tool from contacting (e.g., cutting or moving) the camera. The surgeon may also use a multi-chambered cannula to more effectively use sutures during a surgery. For example, the surgeon may pass one or more sutures through the first chamber in the cannula. The surgeon is then able to use other surgical tools through another chamber in the cannula without those tools becoming entangled in the sutures. In some embodiments, the cannula may be rotated during surgery so that each chamber in the multi-chambered cannula enables access to different interior sections of the patient.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

What is claimed is:

1. A multi-chambered cannula for simultaneously receiving a first tool of a plurality of surgical tools and a second tool of a plurality of surgical tools, the cannula comprising:
a top portion;
a bottom portion;
a first chamber extending between the top portion and the bottom portion, the first chamber being a hollow passage to receive the first surgical tool through the first chamber;
a second chamber extending between the top portion and the bottom portion, the second chamber being a hollow passage to receive the second surgical tool through the second chamber; and
a dividing wall extending from the bottom portion towards the top portion, the dividing wall at least partially separating the first chamber from the second chamber to enable the first surgical tool to pass through the first chamber while the second surgical tool passes simultaneously and separately through the second chamber.

2. The multi-chambered cannula of claim 1, further comprising a dam.

3. The multi-chambered cannula of claim 2, wherein the dam is incorporated into the top portion of the cannula and extends over the first chamber.

4. The multi-chambered cannula of claim 3, wherein the dam further extends over the second chamber.

5. The multi-chambered cannula of claim 3, wherein the dividing wall extends upwards from the bottom portion of the cannula to create a seal with a lower surface of the dam.

6. The multi-chambered cannula of claim 1, wherein the first chamber has a first cross-section and the second chamber has a second cross-section, the first cross-section and the second cross-section being non-congruent.

7. The multi-chambered cannula of claim 6, wherein a shape of a concave surface of the second chamber matches a shape of a convex surface of the first chamber.

8. The multi-chambered cannula of claim 7, wherein the first chamber has a cross-sectional area, the second chamber has a cross-sectional area, and the cross-sectional area of the first chamber is larger than the cross-sectional area of the second chamber.

9. The multi-chambered cannula of claim 1, further comprising a dam that includes a flexible opening configured to resile against a surgical instrument inserted through the flexible opening to create a seal.

10. The multi-chambered cannula of claim 1, further comprising a portal in fluid communication with the first chamber and with the second chamber.

11. The multi-chambered cannula of claim 10, wherein the portal is in fluid communication with the first chamber and with the second chamber through an aperture, and wherein the dividing wall is located adjacent to the aperture to divide fluid passing through the aperture into the first chamber and into the second chamber.

12. A multi-chambered cannula for receiving a first tool of a plurality of surgical tools and a second tool of a plurality of surgical tools, the cannula comprising:
an outer wall unitarily formed of a single material that defines a passage between a top surface of a cannula and a bottom surface of the cannula;
a first channel within the passage, the first channel being configured to enable a first surgical tool to pass through the first channel;
a second channel within the passage, the second channel being configured to enable a second surgical tool to pass through the second channel; and
a dividing wall within the passage that at least partially isolates the first channel from the second channel in order to enable the separate and simultaneous passing of the first surgical tool through the first channel and the second surgical tool through the second channel.

13. The multi-chambered cannula of claim 12, wherein the first channel is configured to receive a suture, the first channel has a cross-sectional area, the second channel has a cross-sectional area that is larger than the cross-sectional area of the first channel, and a width of the cannula measured between opposing sections of an inner surface of the cannula is between 4 and 30 mm.

14. The multi-chambered cannula of claim 13, wherein the width of the cannula is between 4 and 6 mm.

15. The multi-chambered cannula of claim 12, further comprising:
a third channel within the passage, the third channel being configured to enable the surgeon to store a third surgical tool in the third channel.

16. A medical device configured to simultaneously accommodate sutures and other surgical instruments, the medical device comprising:
a cannula formed of a resilient material, the cannula including a channel extending between a top surface of the cannula and a bottom surface of the cannula; and
a dividing wall placed within the channel to divide the channel into a first compartment and a second compartment, wherein the first compartment has a first cross-sectional size configured to accommodate sutures and the second compartment has a second cross-sectional size configured to accommodate another surgical instrument.

17. The medical device of claim 16, further comprising a dam located proximate to the top surface of the cannula.

18. The medical device of claim 17, wherein the dam seals an upper portion of both the first and second compartments, wherein the dam is formed of a resilient material and includes a first perforation located above the first compartment and a second perforation located above the second compartment, and wherein the dam is configured to resile against surgical instruments placed through the first and second perforations to create seals.

19. The medical device of claim 16, wherein a ratio of the first cross-sectional size to the second cross-sectional size ranges from 1:1 to 20:1.

20. The medical device of claim 16, wherein a cross-sectional profile of the second chamber is configured to match a particular surgical instrument.

21. The medical device of claim 16, wherein the dividing wall is a removeable dividing wall adapted to be selectively secured to the cannula.

22. The medical device of claim 16, wherein an outer surface of the cannula has an outer profile configured to continuously contact an outer surface of a surgical incision in order to reduce fluid loss along the outer surface of the cannula.

23. The medical device of claim 16, wherein the first compartment is a blind passage.

24. A multi-chambered cannula for simultaneously receiving a first tool of a plurality of surgical tools and a second tool of a plurality of surgical tools, the cannula comprising:
a top portion;
a bottom portion;
a first chamber extending between the top portion and the bottom portion, the first chamber being a hollow passage to receive the first surgical tool through the first chamber;
a second chamber extending between the top portion and the bottom portion, the second chamber being a hollow passage to receive the second surgical tool through the second chamber; and
a means for separating the first chamber from the second chamber to enable the first surgical tool to pass through the first chamber while the second surgical tool passes simultaneously and separately through the second chamber.
